SEQUENCE LISTING (12 sequences)

SEQ ID NO: 1 - DNA, 49 bp, Artificial Sequence
  Synthetic PCR Primer
tatgcatgcaccatcatcatcaccatggtaattacgaattactgaccac

SEQ ID NO: 2 - DNA, 30 bp, Artificial Sequence
  Synthetic PCR Primer
tatggatccttatccttttacgcacaccac

SEQ ID NO: 3 - DNA, 30 bp, Artificial Sequence
  Synthetic PCR Primer
tatgcatgctaaaaaggatgattgcgctgg

SEQ ID NO: 4 - DNA, 30 bp, Artificial Sequence
  Synthetic PCR Primer
tatggatccttcaatgctcacccgcgttac

SEQ ID NO: 5 - DNA, 1511 bp, Pyrococcus horikoshii
gcatgctgagctctcatcaccatcaccatcacagctctggtgaaaatctgtattttcagatggttgtgccactgaaacgtatcgataaaattcgctgggaaatcccgaaattcgacaagcgtatgcgcgtgcctgggcgtgtttacgcggatgaagtattattggaaaaaatgaaaaatgaccgcaccttggagcaggcgaccaacgtggcgatgttgccaggaatctacaaatattcgattgtgatgccagacggccaccagggctacggcttccccattggcggagtggcggccttcgatgttaaagaaggtgttatcagtccgggcggtatcggatatgacatcaattgtggtgtgcgcctcatccgtactaatctgacggaaaaagaagttcgtccgcgcattaagcagctggtggatacgctgtttaaaaatgttccatccggtgtaggctctcagggccgtattaaactgcattggacgcagattgatgacgtactcgttgatggtgctaaatgggccgttgataacggttatggttgggaacgtgatcttgaacgtttggaggaaggtggccgcatggagggcgccgatccggaagccgtgtcccaacgcgcgaaacagcgcggcgctccacagctgggttctctgggcagtggcaatcactttcttgaagttcaggttgtggataaaattttcgacccggaggtcgccaaagcgtacggtctgtttgaaggccaagtagtagtcatggtgcataccggcagtcgtggtctgggtcatcaagtggcgagtgactaccttcgcatcatggagcgcgccattcgcaaatatcgcattccgtggccggatcgtgagttagtttcagtacccttccagagcgaagaaggtcagcgctattttagcgcgatgaaagcagccgcaaatttcgcctgggccaatcgccaaatgattacgcattgggttcgtgaatccttccaagaggtctttaaacaagatcctgaaggcgatctgggcatggatatcgtgtatgatgttgcccacaacattggtaaagtagaggaacatgaggtcgacggcaaacgtgtcaaagtaattgtacatcgtaaaggcgccacacgcgcgtttccgccgggtcatgaagcggtgccgcgcctgtatcgcgacgtaggccagcccgtactgattccgggttcgatggggaccgcctcttatatcctcgccggcaccgaaggcgcgatgaaagagaccttcggctccacctgtcacggggctgggcgtgttctgtctcgtaaagcggcaacgcgtcaatatcgcggtgaccgcattcgtcaggagctccttaatcgcggtatttacgtgcgtgccgcttctatgcgtgttgtggcggaggaagctccaggggcgtataaaaacgttgacaatgtggttaaagtggttagcgaagcaggcattgccaaactcgttgcacgtatgcgcccgatcggtgtagcgaaaggctaaagatct

SEQ ID NO: 6 - RNA, 10 nt, Artificial Sequence
  Synthetic RNA fragment
aaauaacaaa

SEQ ID NO: 7 - RNA, 14 nt, Artificial Sequence
  Synthetic RNA fragment
uaaauaacaaagau

SEQ ID NO: 8 - DNA, 10 bp, Artificial Sequence
  Synthetic DNA/RNA hybrid
aaauaacaaa

SEQ ID NO: 9 - protein, 142 aa, Pyrococcus horikoshii
MKKWEHYEHTADIGIRGYGDSLEEAFEAVAIALFDVMVNVNKVEKKEVREIEVEAEDLEALLYSFLEELLVIHDIEGLVFRDFEVKIERVNGKYRLRAKAYGEKLDLKKHEPKEEVKAITYHDMKIERLPNGKWMAQLVPDI

SEQ ID NO: 10 - protein, 481 aa, Pyrococcus horikoshii
MVVPLKRIDKIRWEIPKFDKRMRVPGRVYADEVLLEKMKNDRTLEQATNVAMLPGIYKYSIVMPDGHQGYGFPIGGVAAFDVKEGVISPGGIGYDINCGVRLIRTNLTEKEVRPRIKQLVDTLFKNVPSGVGSQGRIKLHWTQIDDVLVDGAKWAVDNGYGWERDLERLEEGGRMEGADPEAVSQRAKQRGAPQLGSLGSGNHFLEVQVVDKIFDPEVAKAYGLFEGQVVVMVHTGSRGLGHQVASDYLRIMERAIRKYRIPWPDRELVSVPFQSEEGQRYFSAMKAAANFAWANRQMITHWVRESFQEVFKQDPEGDLGMDIVYDVAHNIGKVEEHEVDGKRVKVIVHRKGATRAFPPGHEAVPRLYRDVGQPVLIPGSMGTASYILAGTEGAMKETFGSTCHGAGRVLSRKAATRQYRGDRIRQELLNRGIYVRAASMRVVAEEAPGAYKNVDNVVKVVSEAGIAKLVARMRPIGVAKG

SEQ ID NO: 11 - protein, 408 aa, Escherichia coli
MNYELLTTENAPVKMWTKGVPVEADARQQLINTAKMPFIFKHIAVMPDVHLGKGSTIGSVIPTKGAIIPAAVGVDIGCGMNALRTALTAEDLPENLAELRQAIETAVPHGRTTGRCKRDKGAWENPPVNVDAKWAELEAGYQWLTQKYPRFLNTNNYKHLGTLGTGNHFIEICLDESDQVWIMLHSGSRGIGNAIGTYFIDLAQKEMQETLETLPSRDLAYFMEGTEYFDDYLKAVAWAQLFASLNRDAMMENVVTALQSITQKTVRQPQTLAMEEINCHHNYVQKEQHFGEEIYVTRKGAVSARAGQYGIIPGSMGAKSFIVRGLGNEESFCSCSHGAGRVMSRTKAKKLFSVEDQIRATAHVECRKDAEVIDEIPMAYKDIDAVMAAQSDLVEVIYTLRQVVCVKG

SEQ ID NO: 12 - DNA, 453 bp, Pyrococcus horikoshii
atgcatcaccatcaccatcatggcatgaaaaagtgggaacattatgaacacaccgcagatattggtatccgtggctatggtgattcactggaagaggcgtttgaggccgtagctatcgcgctgttcgacgtcatggtgaatgttaataaggttgaaaaaaaagaggttcgcgaaattgaggtagaagcggaagatctggaagcgttactgtatagctttttggaagaacttttagtgattcacgatattgagggcttagtcttccgcgattttgaggtgaaaattgaacgcgttaatggcaaataccgtttacgtgccaaagcgtacggcgaaaaactggatcttaaaaaacacgaacctaaagaagaagtaaaagccattacgtaccatgatatgaaaattgagcgcctcccgaacggcaaatggatggcccaattagtaccggatatttaa

(19) United States
(12) Patent Application Publication
(10) Pub. No.: US 2013/0203635 A1
(43) Pub. Date: Aug. 8, 2013

(54) NUCLEIC ACID LIGATION METHOD

(71) Applicants: Ronald T. Raines, Madison, WI (US); Kevin K. Desai, Madison, WI (US)

(72) Inventors: Ronald T. Raines, Madison, WI (US); Kevin K. Desai, Madison, WI (US)

(21) Appl. No.: 13/760,920

(22) Filed: Feb. 6, 2013

Related U.S. Application Data
(60) Provisional application No. 61/595,482, filed on Feb. 6, 2012.

(51) Int. Cl. C12P 19/34

(52) U.S. Cl. CPC C12P 19/34
USPC 506/26; 435/91.52

(57) ABSTRACT

Methods and kits for covalently joining a 3′ nucleic acid fragment having a 5′-hydroxyl terminus to a 5′ nucleic acid fragment having a 3′-phosphate terminus are disclosed. The methods include the step of contacting the 3′-phosphate terminus of a first nucleic acid molecule and the 5′-hydroxyl terminus of a second nucleic acid molecule with an isolated 2′,3′-cyclic phosphate RNA ligase (RtcB) and a purine triphosphate in the presence of manganese (II) ion, whereby the 3′-phosphate terminus of the first nucleic acid molecule and the 5′-hydroxyl terminus of the second nucleic acid molecule are covalently joined.

Although the purine triphosphate used is generally GTP or dGTP, if the method is performed in the presence of an Archease, any purine triphosphate may be used. Accordingly, the disclosed kits include isolated RtcB, along with a purine triphosphate and/or an isolated Archease. Such methods and kits can be used to tag or ligate DNA or RNA on its 3′-phosphate terminus, as long as the terminal residue at the 3′-phosphate terminus is an RNA nucleotide.

### CROSS-REFERENCE TO RELATED APPLICATION

This application claims the benefit of U.S. Provisional Application No. 61/595,482 filed on Feb. 6, 2012, which is incorporated by reference herein in its entirety.

### STATEMENT REGARDING FEDERALLY SPONSORED RESEARCH OR DEVELOPMENT

This invention was made with government support under CA073808 awarded by the National Institutes of Health. The government has certain rights in the invention.

### FIELD OF THE INVENTION

This disclosure relates generally to methods for ligating nucleic acid molecules. In particular, the present disclosure encompasses methods of and kits for directly ligating a 3′-phosphate terminus of a nucleic acid molecule to a 5′-hydroxyl terminus of the same or of a different nucleic acid molecule.

### BACKGROUND OF THE INVENTION

The ligation of RNA molecules entails the joining of activated phosphate and hydroxyl termini to generate a phosphodiester bond. Known enzymes catalyze the ligation of RNA using two distinct pairs of termini: (1) 3′-hydroxyl+5′-phosphate, or (2) 2′,3′-cyclic phosphate+5′-hydroxyl.

2′,3′-Cyclic phosphate RNA ligase (RtcB) has been identified as the enzyme responsible for catalyzing the cofactor independent, direct ligation of 2′,3′-cyclic phosphate and 5′-hydroxyl RNA termini. This activity is essential in archaea and metazoa for the ligation of transfer RNA (tRNA) molecules after intron removal by the tRNA splicing endonuclease. The intron removal reaction proceeds in two separate steps: cleavage of RNA to generate fragments with 2′,3′-cyclic phosphate and 5′-OH termini, and hydrolysis of the 2′,3′-cyclic phosphate to form a 3′-phosphate (3′-P).

The fate of 3′-P RNA termini in tRNA splicing intermediates is not well understood, but they are presumed to be recyclized by RNA phosphate cyclase (RtcA). RtcA is highly conserved in all three domains of life and is also postulated to function in the cyclization of the 3′-P of spliceosomal U6 snRNA. Synthesis of 2′,3′-cyclic phosphate termini by the ATP-dependent RtcA occurs in three nucleotidyl transfer steps: (1) reaction of ATP with an active-site histidine residue to form a covalent enzyme—AMP intermediate and release PPᵢ, (2) transfer of the AMP moiety to the terminal 3′-P to form an RNA—adenylylate intermediate, and (3) attack by the terminal 2′-OH on the adenylylated 3′-P to form the 2′,3′-cyclic phosphate product and release AMP. This cyclization of 3′-P RNA termini is known in the art to be a prerequisite for ligation to 5′-OH RNA termini.

In general, when a ribonuclease cleaves an RNA molecule, the cleavage generates a 2′,3′ cyclic phosphate as an intermediate, and the final cleavage products have a 3′-phosphate terminus on one strand, and a 5′-hydroxyl terminus on another strand. An example of this was described above in the context of the removal of transfer RNA (tRNA) introns by tRNA splicing endonuclease. Fragments having 3′-phosphate and 5′-hydroxyl termini are also generated by bovine pancreatic ribonuclease (RNase A), the archetypal acid-base catalyst. This specific termini pair combination in the fragments produced by ribonuclease cleavage of RNA (3′-phosphate+5′-hydroxyl) cannot be directly ligated using known methods. Accordingly, there is a need in the art for methods and kits for directly ligating nucleic acid fragments having a 3′-phosphate to nucleic acid fragments having a 5′-hydroxyl terminus.

### SUMMARY OF THE INVENTION

RNA ligases are only known to accept strands with two distinct pairs of termini. We have surprisingly determined that a third pair of ligase-competent RNA termini can be successfully ligated using the RNA ligase RtcB. Specifically, RtcB catalyzes the unprecedented purine triphosphate-dependent ligation of RNA with 3′-phosphate and 5′-hydroxyl termini. Reactions with analogues of RNA and GTP suggest a mechanism in which RtcB heals the 3′-phosphate terminus by forming a 2′,3′-cyclic phosphate before joining it to the 5′-hydroxyl group of a second RNA strand. Thus, RtcB can ligate RNA cleaved by ribonucleases, which generate 2′,3′-cyclic phosphate and then 3′-phosphate termini on one strand, and a 5′-hydroxyl terminus on another strand.

Furthermore, we have found that the method can be generalized to either DNA or RNA, as long as the single nucleotide at the 3′-phosphate terminus of the 5′ fragment is an RNA nucleotide.

In one aspect, the disclosure encompasses a method for covalently joining a first nucleic acid molecule (the 5′ fragment) and a second nucleic acid molecule (the 3′ fragment). The first nucleic acid molecule includes a 3′-phosphate terminus, and the nucleotide at the 3′-phosphate terminus is an RNA nucleotide. The second nucleic acid molecule comprises a 5′-hydroxyl terminus.

The method is performed by contacting the 3′-phosphate terminus of the first nucleic acid and the 5′-hydroxyl terminus of the second nucleic acid molecule with a 2′,3′-cyclic phosphate RNA ligase (RtcB) in the presence of manganese (II) ion (Mn²⁻) and a purine triphosphate. Under these conditions, the 3′-phosphate terminus of the first nucleic acid molecule and the 5′-hydroxyl terminus of the second nucleic acid molecule are covalently joined.

In some embodiments, one or more of the RtcB, the first nucleic acid molecule, the second nucleic acid molecule, and the purine triphosphate are isolated. In some embodiments, the method is performed in vitro.

In some embodiments, the purine triphosphate is guanosine-5′-triphosphate (GTP) or deoxyguanosine-5′-triphosphate (dGTP). In some such embodiments, the purine triphosphate is GTP.

In some embodiments, the method is performed in the presence of an Archease enzyme. Although the method generally requires GTP or dGTP, in the presence of an Archease, any purine triphosphate, including adenosine-5′-triphosphate (ATP), may be used.

In some embodiments, the method is performed in the absence of an RNA phosphate cyclase (RtcA). Optionally, the method may be performed in the presence of polyethylene glycol (PEG), which increases the efficiency of the ligation reaction.

In some embodiments, the first nucleic acid molecule is an RNA molecule or a DNA molecule having a single RNA nucleotide at the 3′-phosphate terminus. In some embodiments, the second nucleic acid molecule is an RNA molecule or a DNA molecule.

The ligation product formed by practicing the method may be without limitation an RNA molecule, a DNA molecule having a single RNA nucleotide, or a RNA/DNA hybrid molecule. The first and second nucleic acid molecules are not necessarily separate fragments, but may be a single molecule having the two required termini. In such an embodiment, a circular nucleic acid molecule may be formed as the ligation product.

Optionally, either nucleic acid (and preferably, the second nucleic acid molecule) may include a detection moiety, including without limitation a fluorophore or a radiolabel.

In certain embodiments, the method may be performed at relatively high temperatures. As non-limiting examples, the method is performed at a temperature above 50° C., or at a temperature above 60° C.

In another aspect, the disclosure encompasses a method for making a library of nucleic acid fragments. In performing the method, a first nucleic acid molecule including a 3′-phosphate terminus wherein the nucleotide at the 3′-phosphate terminus is an RNA nucleotide is contacted with a plurality of second nucleic acid molecules having a 5′-hydroxyl terminus with a 2′,3′-cyclic phosphate RNA ligase (RtcB), manganese (II) ion (Mn²⁺), and a purine triphosphate. The resulting ligation reaction produces a library of nucleic acid fragments. In the absence of an Archease, the purine triphosphate may be guanosine-5′-triphosphate (GTP) or deoxyguanosine-5′-triphosphate (dGTP).

In certain embodiments, the method is performed in the presence of an Archease. In some such embodiments, the purine triphosphate may be GTP, dGTP, or adenosine-5′-triphosphate (ATP).

In a third aspect, the disclosure encompasses a kit for performing the method outlined above. The kit includes an isolated 2′,3′-cyclic phosphate RNA ligase (RtcB) and either (1) an isolated guanosine-5′-triphosphate (GTP) or deoxyguanosine-5′-triphosphate (dGTP); or (2) an isolated Archease. Optionally, the kit may also include a composition containing the manganese (II) ion. In embodiments where the kit includes an isolated Archease, the kit may further include an isolated purine triphosphate. Non-limiting examples of purine triphosphates that may be included are GTP, dGTP, or adenosine-5′-triphosphate (ATP).

In some embodiments, the kit may further include a nucleic acid molecule having a 5′-hydroxyl terminus. Optionally, the nucleic acid molecule may include a detection moiety for labeling nucleic acids, including without limitation a fluorophore or a radiolabel. In some embodiments, the nucleic acid molecule included with the kit is an RNA molecule or a DNA molecule.

These and other features of the present invention will become apparent to the skilled artisan from the following detailed description considered in conjunction with the accompanying drawings.

### BRIEF DESCRIPTION OF THE DRAWINGS

FIG. 1. E. coli RtcB catalyzed GTP-dependent ligation of 3′-P and 5′-OH RNA termini. (A) RNA ligation substrate that mimics a broken tRNA anti-codon. FAM label allows visualization in a urea-polyacrylamide gel. Ligation of 3′-P and 5′-OH termini generates a 35-nt RNA, whereas unligated substrate appears as a 17-nt band. (B) Cofactor- and metal-dependence of ligation. Reactions contain manganese, except in the “—Mn²⁺” lane. The lane labeled “C78A” is a ligation reaction performed with RtcB that has a C78A substitution within the predicted metal binding site. (C) Ligation assays using GTP analogues as cofactors. (D) GTP concentration-dependence of ligation, allowing estimation of a GTP K_{M} that is <16 μM. (E) RNA termini specificity of RtcB. Modifications to the ligatable ends of the tRNA mimic substrate were made as indicated and tested in ligation reactions.

FIG. 2. P. horikoshii RtcB catalyzed ligation of 3′-P and 5′-OH RNA termini. P. horikoshii RtcB requires Mn²⁺ and GTP for ligation. Omission of Mn²⁺ or replacement with Zn²⁺ did not allow ligation to proceed. The lane labeled “2′-deoxy” is a ligation reaction using the tRNA mimic substrate but with a 3′-P/2′-H termini on its 5′ fragment. The lane labeled “C98A” is a ligation reaction with RtcB that has a C98A substitution within the predicted metal-binding site.

FIG. 3. Nucleotide sequence of synthetic P. horikoshii rtcB gene with optimized codons for expression in E. coli (SEQ ID NO:5). The translation start and stop codons are underlined.

FIG. 4. Effect of RNA phosphate cyclase (RtcA), GTP, ATP, and Mg²⁺ on catalysis of RNA ligation by RtcB. (A) Effect of RNA phosphate cyclase (RtcA) and GTP. Lanes 1 and 2 are reaction mixtures (50 μL) consisting of 50 mM Tris-HCl buffer, pH 7.4, containing NaCl (200 mM), MgCl₂ (2 mM), DTT (2 mM), ATP (0.3 mM), the 5′ RNA fragment containing a 3′-P (200 pmol), and E. coli RtcA (1 μM). Reaction mixtures were incubated at 37° C. for 30 min prior to the addition of MnCl₂ (2 mM), the 3′ RNA fragment (200 pmol), and E. coli RtcB (1 μM). Lanes 1 and 2 are identical reaction mixtures in which the 5′ RNA fragment was incubated with RtcA prior to the addition of RtcB. Lane 3 is a reaction mixture without RtcA, but with GTP (0.3 mM). Lane 4 is a reaction mixture without RtcB. Lane 5 is a reaction mixture without RtcA and RtcB. (B) Effect of GTP, ATP, and Mg²⁺. Lane 1 is a reaction mixture (50 μL) consisting of 50 mM Tris-HCl buffer, pH 7.4, containing NaCl (200 mM), GTP (0.3 mM), ATP (0.3 mM), MnCl₂ (0.5 mM), MgCl₂ (1 mM), each RNA fragment (40 pmol), and E. coli RtcB (1 μM). Lane 2 is a reaction mixture without MgCl₂. Lane 3 is a reaction mixture without GTP. Lane 4 is a reaction mixture without ATP. Lane 5 is a reaction mixture without RtcB.

FIG. 5. Polyacrylamide electrophoresis gel showing labeled substrate and ligation product from an incubating reaction mixture at 25° C. (leftmost lane), 37° C. (center lane), and 70° C. (rightmost lane).

FIG. 6. Polyacrylamide electrophoresis gel showing labeled substrate and four different ligation products making up an RNA fragment library generated using the disclosed method.

FIG. 7. RNA ligation assays using variants of RtcB in which GMP-interacting residues have been replaced with alanine RtcB is also able to use dGTP as a cofactor, though the ligation efficiency is reduced greatly. The ligase substrates are two 10-nt single-stranded RNAs. The 5′ RNA fragment is labeled with FAM to allow visualization in a urea-polyacrylamide gel; the 3′ RNA fragment has hydroxyl groups at each terminus.

FIG. 8. Urea-polyacrylamide gel showing the ligation of a DNA/RNA hybrid by RtcB. Reaction mixtures were 50 mM Tris-HCl buffer, pH 7.4, containing NaCl (300 mM), MnCl₂ (0.25 mM), GTP (100 μM), DNA/RNA fragments (100 pmol of FAM-5′-d(AAAUAACAA)A-3′-P (SEQ ID NO:8) and 5′-AAAUAACAAA-3′(SEQ ID NO:6)), and P. horikoshii RtcB (5 μM).

FIG. 9. Urea-polyacrylamide gel showing the effect of Archease on catalysis of RNA ligation by RtcB. Reaction mixtures (50 μL) were 50 mM Tris-HCl buffer, pH 7.4, containing NaCl (300 mM), MnCl₂ (0.25 mM), GTP (100 μM), RNA fragments (100 pmol of FAM-5′-AAAUAACAAA-3′-P (SEQ ID NO:6) and 5′-AAAUAACAAA-3′ (SEQ ID NO:6)), P. horikoshii RtcB (5 μM), and Archease (various concentrations). (A) 0-5 μM Archease. (B) 0-2 μM Archease.

FIG. 10. Urea-polyacrylamide gel showing the effect of Archease on the loading of a catalysis of RNA ligation by RtcB. Reaction mixtures (50 μL) were 50 mM Tris-HCl buffer, pH 7.4, containing NaCl (300 mM), MnCl₂ (0.25 mM), GTP or dGTP (100 μM), RNA fragments (100 pmol of FAM-5′-AAAUAACAAA-3′-P (SEQ ID NO:6) or 2′-F,3′-P, and 5′-AAAUAACAAA-3′ (SEQ ID NO:6)), P. horikoshii RtcB (5 μM). (A) No Archease. (B) 0.25 μM Archease.

FIG. 11. Urea-polyacrylamide gel showing the effect of Archease on the cofactor specificity of RtcB. Reaction mixtures (50 μL) were 50 mM Tris-HCl buffer, pH 7.4, containing NaCl (300 mM), MnCl₂ (0.25 mM), NTP (100 μM), RNA fragments (100 pmol of FAM-5′-AAAUAACAAA-3′-P (SEQ ID NO:6) and 5′-AAAUAACAAA-3′(SEQ ID NO:6)), P. horikoshii RtcB (5 μM). (A) No Archease. (B) 0.25 μM Archease.

FIG. 12. DNA sequence of P. horikoshii archease with codons optimized for expression in E. coli (SEQ ID NO:12). The translation start and stop codons are underlined.

### DETAILED DESCRIPTION OF THE INVENTION

### I. IN GENERAL

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by any later-filed nonprovisional applications.

As used herein and in the appended claims, the singular forms “a”, “an”, and “the” include plural reference unless the context clearly dictates otherwise. As well, the terms “a” (or “an”), “one or more” and “at least one” can be used interchangeably herein. The terms “comprising”, “including”, and “having” can be used interchangeably.

Unless defined otherwise, all technical and scientific terms used herein have the same meanings as commonly understood by one of ordinary skill in the art. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### II. THE INVENTION

The inventors have recently demonstrated a new method for ligating nucleic acid molecules. Known enzymes catalyze the ligation of RNA using two distinct pairs of termini: (1) 3′-hydroxyl+5′-phosphate, or (2) 2′,3′-cyclic phosphate +5′-hydroxyl. The inventors have discovered that the RtcB enzyme, which is found in forms of life from E. coli to humans (and is well-characterized in numerous species), catalyzes the ligation of RNA using a third pair of termini: (3) 3′-phosphate+5′-hydroxyl.

These three ligation reactions are depicted in the following scheme, with the previously known reactions illustrated in the first two lines, and the reaction of the present method illustrated in the third line:

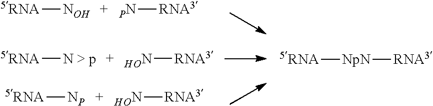

In some embodiments, the newly disclosed ligation reaction relies on the presence of GTP, which is cleaved to GMP and PPᵢ during catalysis. Furthermore, the disclosed ligation reaction is most efficient in the presence of manganese (II) ion.

In some embodiments, the newly disclosed ligation reaction relies on the presence of an Archease co-factor. Archeases have been found and characterized across a variety of species from all three domains of life. Although the precise function of Archeases is not well-understand, Archease genes are generally located adjacent to genes encoding proteins involved in DNA or RNA processing. Thus, they are thought to function as chaperones or as co-factors involved in nucleic acid processing. In such embodiments, the purine triphosphate used is not limited to GTP (or dGTP), and ATP may be used instead.

Although the RtcB enzyme was known before the present invention, the present inventors were the first to discover its ability to ligate a 3′-phosphate terminus to a 5′-hydroxyl terminus. This discovery is important for practical applications, because this pair of termini is the easiest to access by both synthetic chemistry and the enzyme-catalyzed or base-catalyzed cleavage of RNA. For example, ribonucleases produce these termini upon RNA cleavage.

The disclosed method allows RNA or DNA to be tagged or ligated on its 3′-phosphate terminus. Although the terminal residue at the 3′-phosphate terminus must be an RNA nucleotide; all the other nucleotides in the nucleic acid molecules that are ligated may be either DNA or RNA. The skilled artisan could readily “cap” a DNA fragment with a single RNA nucleotide to practice the method using DNA molecules.

Thus, the invention encompasses methods and kits for joining any nucleic acid fragment (perhaps labeled with a fluorophore or radiolabel) to another RNA or DNA molecule having a 3′-phosphate terminus. One potential application of the method is in the tagging or labeling of RNA or DNA molecules. Other applications are also within the scope of the disclosed method, including without limitation making RNA-DNA hybrids and making circular RNA or DNA.

The following examples are offered for illustrative purposes only, and are not intended to limit the scope of the present invention in any way. Indeed, various modifications of the disclosed method in addition to those shown and described herein will become apparent to those skilled in the art from the foregoing description and the following examples and fall within the scope of the appended claims.

### III. EXAMPLES

### Example 1

### RtcB Catalyzes the GTP-Dependent Ligation of RNA with 3′-Phosphate and 5′-Hydroxyl Termini

Summary.

RNA ligases are only known to accept strands with two distinct pairs of termini. In this example, we report on a third pair of ligase-competent RNA termini. The RNA ligase RtcB is conserved in all domains of life, and is essential for tRNA maturation in archaea and metazoa. We find that bacterial and archaeal RtcB catalyze the unprecedented GTP-dependent ligation of RNA with 3′-phosphate and 5′-hydroxyl termini. Reactions with analogues of RNA and GTP suggest a mechanism in which RtcB heals the 3′-phosphate terminus by forming a 2′,3′-cyclic phosphate before joining it to the 5′-hydroxyl group of a second RNA strand. Thus, RtcB can ligate RNA cleaved by ribonucleases, which generate 2′,3′-cyclic phosphate and then 3′-phosphate termini on one strand, and a 5′-hydroxyl terminus on another strand.

Introduction.

The ligation of RNA molecules entails the joining of activated phosphate and hydroxyl termini to generate a phosphodiester bond (1, 2). 2′,3′-Cyclic phosphate RNA ligase (RtcB) has been identified as the enzyme responsible for catalyzing the cofactor-independent, direct ligation of 2′,3′-cyclic phosphate and 5′-OH RNA termini (3-5). This activity is essential in archaea and metazoa for the ligation of transfer RNA (tRNA) molecules after intron removal by the tRNA splicing endonuclease (6-8). That endonuclease generates fragments analogous to those of the archetypal acid-base catalyst: bovine pancreatic ribonuclease (RNase A) (9-12). These reactions proceed in two separate steps: cleavage of RNA to generate fragments with 2′,3′-cyclic phosphate and 5′-OH termini, and hydrolysis of the 2′,3′-cyclic phosphate to form a 3′-phosphate (3′-P) (13, 14).

The fate of 3′-P RNA termini in tRNA splicing intermediates is not well understood, but they are presumed to be recyclized by the enigmatic RNA phosphate cyclase (RtcA) (15). RtcA is highly conserved in all three domains of life and is also postulated to function in the cyclization of the 3′-P of spliceosomal U6 snRNA (16). Synthesis of 2′,3′-cyclic phosphate termini by the ATP-dependent RtcA occurs in three nucleotidyl transfer steps: (1) reaction of ATP with an active-site histidine residue to form a covalent enzyme-AMP intermediate and release PPᵢ, (2) transfer of the AMP moiety to the terminal 3′-P to form an RNA adenylylate intermediate, and (3) attack by the terminal 2′-OH on the adenylylated 3′-P to form the 2′,3′-cyclic phosphate product and release AMP (17). This cyclization of 3′-P RNA termini is thought to be a prerequisite for ligation to 5′-OH RNA termini.

Procedures, Results, and Discussion.

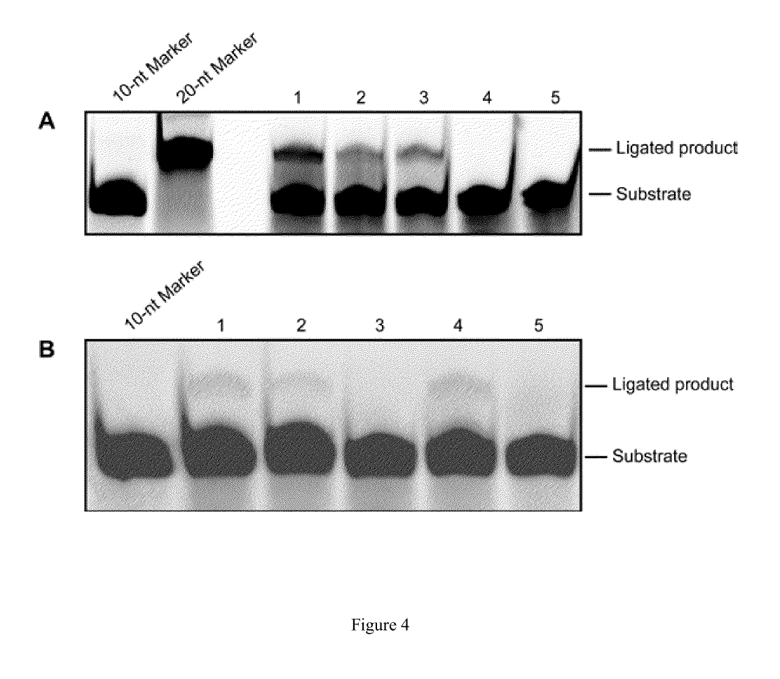

We have discovered that Escherichia coli RtcB ligase (SEQ ID NO:11; UniProt Accession No. P46850) can accept 3′-P RNA termini as its substrate. To assay for ligation, we initially used two single-stranded RNA (ssRNA) oligos that were each 10 nucleotides in length. The 5′ RNA fragment was labeled with 6-carboxyfluorescein (FAM) at its 5′ end and phosphorylated at its 3′ end. The 3′ RNA fragment had hydroxyl groups at each end. We intended to convert the 3′-P into a 2′,3′-cyclic phosphate by incubation with RtcA and ATP, so as to produce a suitable substrate for RtcB. Surprisingly, we found that prior cyclization of the 3′-P was not necessary for ligation to a 5′-OH RNA termini, as long as GTP was included in reaction mixtures (FIG. 4). This result was found even with RtcB produced in an rtcA deletion strain of E. coli, eliminating the possibility of inadvertent contamination.

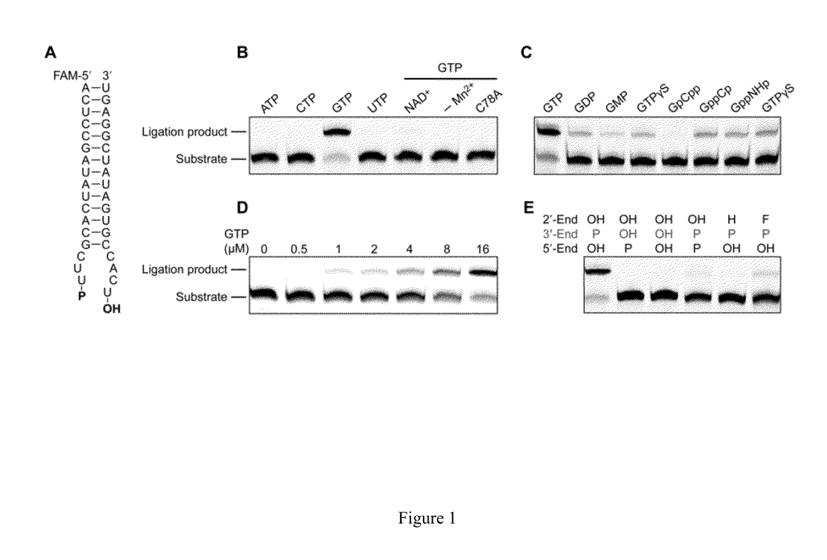

To explore this reactivity further, we used a substrate that mimics the broken tRNA anticodon stem-loop of yeast tRNA^{Glu(UUC)} (FIG. 1A), which was ligated more efficiently by RtcB than was ssRNA because of the proximity of its 3′-P and 5′-OH termini. We found that ATP, CTP, UTP, or NAD⁺ were unable to substitute for GTP (FIG. 1B). Ligation was also strictly dependent on Mn²⁺, and a C78A substitution (Cys⁷⁸→Ala⁷⁸) within a predicted metal-binding site abolished ligase activity, as reported previously for the ligation of RNA substrates bearing 2′,3′-cyclic phosphate and 5′-OH termini (5).

Then, we tested five GTP analogues as cofactors in ligation reactions, and discovered that bond cleavage between the α and β phosphoryl groups of GTP occurs during catalysis by RtcB. These reactions were performed with high concentrations of GTP analogues to ensure saturation of RtcB. Four of the analogues, β,γ-methylene (GppCp), β,γ-imido (GppNHp), γ-thio (GTPγS), and α-thio (GTPαS), enabled ligation, albeit at lower efficiency than did GTP (FIG. 1C). In contrast, α,β-methylene (GpCpp) did not enable the ligation to proceed, suggesting that cleavage of the phosphoanhydride bond between the α and β phosphoryl groups of GTP is critical for the ligation of 3′-P and 5′-OH RNA termini. GDP and GMP allowed for low levels of ligation, which could arise from low amounts of contaminating GTP. Titrations with GTP indicate that RtcB has a K_{M} of less than 16 μM for this cofactor (FIG. 1D). This value is similar to the ATP K_{M} of 20 μM for E. coli RtcA (18) and 6 μM for human RtcA (19).

The GTP-dependent ligation activity of RtcB requires a 3′-P/2′-OH on the 5′ side of the ligation junction and a 5′-OH on the 3′ side. We investigated the RNA termini structure requirements for ligation by modifying the ends of our tRNA mimic substrate. First, all four combinations of either a phosphate or hydroxyl group at each termini were sampled while maintaining a 2′-OH on the 5′ side of the ligation junction. RtcB was found to be highly specific for RNA substrates that have 3′-P and 5′-OH termini (FIG. 1E). Next, the importance of the terminal ribose 2′-OH for ligation was probed by replacing this hydroxyl group with either hydrogen (2′-H) or fluorine (2′-F). The RNA substrate with a 3′-P/2′-H termini produced no observable ligation product, whereas the 3′-P/2′-F substrate yielded only trace product. The terminal ribose of the substrate with a 3′-P/2′-F end will adopt a ring pucker similar to that of the 3′-P/2′-OH substrate (20), but will not allow for phosphate cyclization. These data suggest that the mechanism for ligation involves cyclization of the 3′-P as a prerequisite for ligation to a 5′-OH RNA termini.

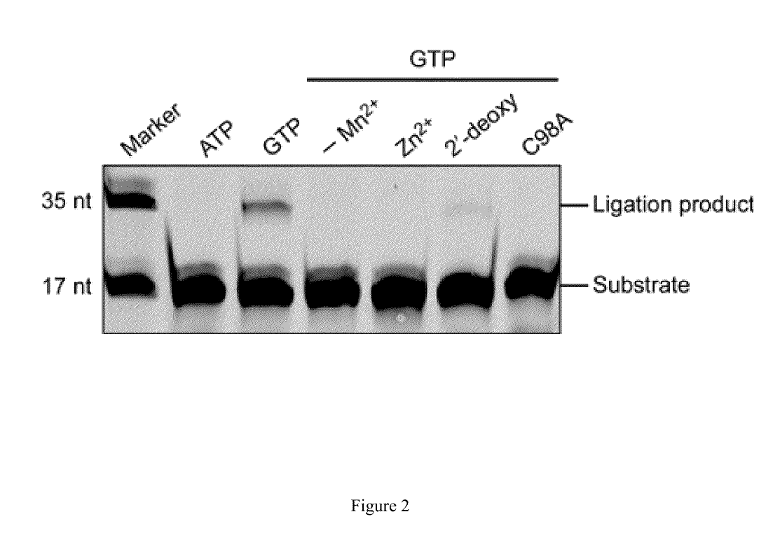

RtcB from the archaeal domain of life likewise catalyzes the GTP-dependent ligation of 3′-P and 5′-OH RNA termini. The crystal structure of RtcB from a hyperthermophilic archaeon, Pyrococcus horikoshii, was determined as part of a structural genomics project (21). This structure reveals a hydrophilic pocket with a predicted metal-binding site consisting of residues Cys98, His203, His234, and His404. The P. horikoshii homologue has 29% amino-acid sequence identity to E. coli RtcB, and 49% identity to the human ligase (HSPC117). To investigate if RtcB from archaea catalyzes the GTP-dependent ligation of 3′-P and 5′-OH RNA termini, we synthesized a P. horikoshii rtcB gene and produced soluble enzyme in E. coli. RtcB from P. horikoshii catalyzed the same reaction as its E. coli homologue and displayed identical cofactor and metal-ion requirements (FIG. 2). The P. horikoshii ligase also required a 2′-OH on the terminal nucleotide of the 5′ RNA fragment. Although RtcB from the archaeon Pyrobaculum aerophilum has been reported to depend on Zn²⁺ for activity (4), we found that Zn²⁺ is unable to replace Mn²⁺ in active P. horikoshii RtcB. We did find that a C98A substitution (Cys⁹⁸→Ala⁹⁸) within the predicted metal-binding site abolishes ligase activity, as reported for P. aerophilum RtcB (4).

We propose a three-step mechanism for the ligation of 3′-P and 5′-OH RNA termini catalyzed by RtcB (Scheme 1). In the activation step, cleavage of the phosphoanhydride bond between the α and βphosphoryl groups of GTP occurs during transfer of GMP to the 3′-P terminus of an RNA strand to form a guanylylate-activated intermediate. ATP-dependent DNA and RNA ligases that catalyze a similar activation of 5′-P RNA termini first form a covalent enzyme-AMP intermediate with an active-site lysine, with subsequent transfer of AMP to the 5′-P termini (1). In the cyclization step of our mechanism, the 2′-OH on the terminal ribose attacks the GMP-activated 3′-P to generate a 2′,3′-cyclic phosphate and release GMP. In the ligation step, the 5′-OH terminus of a second RNA strand attacks the cyclic phosphate to form a 3′,5′-phosphodiester bond. We note that this mechanism accommodates the competency of an RNA strand with a 2′,3′-cyclic phosphate to be a substrate for RtcB in the absence of GTP (3-5).

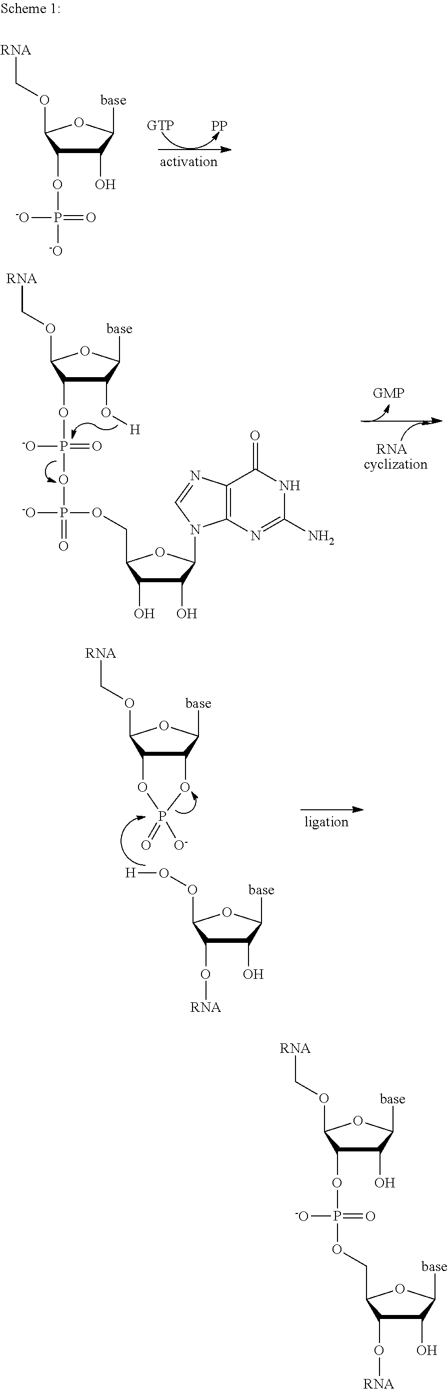

To date, only two other sets of RNA termini have been identified as competent substrates for an RNA ligase. Bacteriophage T4 RNA ligase 1 (Rnl1) and RNA ligase 2 (Rnl2) catalyze the ATP-dependent ligation of 3′-OH and 5′-P RNA termini in three nucleotidyl transfer steps similar to those of DNA ligases (1, 22). All three domains of life possess ligases that accept 2′,3′-cyclic phosphate and 5′-OH RNA termini (4) and that are known to be essential for tRNA maturation and the unconventional splicing of HAC1 mRNA (2). The yeast tRNA ligase is a class I 5′-P RNA ligase and is homologous to T4 Rnl1 (2). The multifunctional yeast ligase is unable to join tRNA exon termini directly, but instead has cyclic phosphodiesterase and polynucleotide kinase activities that yield 3′-OH/2′-P and 5′-P termini. The yeast ligase then seals these ends in an ATP-dependent reaction (23). In plants, a similar mechanism catalyzed by the class II 5′-P ligase forms mature tRNAs (24, 25).

In contrast, archaea and metazoa form mature tRNAs via the direct ligation of 2′,3′-cyclic phosphate and 5′-OH termini in a reaction catalyzed by RtcB (4). This reaction is unique in that it entails nucleophilic attack by a 5′-OH, which has a much higher pKₐ value than does a 3′-OH (26). The presence of RtcB in bacteria is mysterious because group 1 introns in bacterial pre-tRNAs self-splice to form mature tRNAs (27). It has been suggested that E. coli RtcB might serve to protect against damage from stress-induced tRNA endonucleases (5), which cleave tRNA using a mechanism similar to that of RNase A (28). The discovery that RtcB is a multifunctional ligase that can prepare a 3′-P termini for ligation to a 5′-OH, establishes a third pair of RNA termini that are suitable substrates for an RNA ligase (Scheme 2).

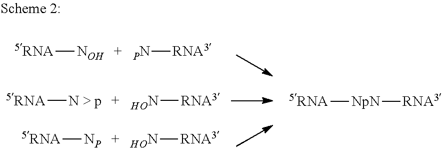

Recently, the human RtcB homologue was purified from HeLa cell extracts using the activity-guided purification of ligase activity (3). A double-stranded RNA (dsRNA) substrate containing 3′-P and 5′-OH termini was used to follow human ligase activity after the dsRNA became linked, apparently after cyclization of the 3′-P by RtcA. The covalent linking of 3′-P and 5′-OH dsRNA, upon incubation with human cell extract, has also been observed in other studies (15, 29). Our data are consistent with RtcB being the sole enzyme responsible for these previous observations. In addition, our findings question the importance of RtcA in maintaining the 2′,3′-cyclic phosphate termini of tRNA splicing intermediates.

A search of the NCBI database reveals that RtcB has no homology to other known RNA or DNA ligases and does not have a predictable GTP-binding site. Furthermore, RtcB does not contain the conserved sequence motif KXXG that defines a superfamily of covalent nucleotidyltransferases, which includes DNA and RNA ligases and mRNA-capping enzymes (1). Future studies will shed light on the GTP binding motif of RtcB and whether its mechanism involves a covalent enzyme-GMP intermediate, in analogy to known nucleic acid ligases. Finally, we note that RtcB is able to repair RNA strands that have been cleaved by RNase A and other pancreatic-type ribonucleases, which generate 2′,3′-cyclic phosphate and then 3′-P termini on one strand, and 5′-OH termini on the other strand (13, 14). Moreover, the ease of synthesizing RNA strands with 3′-P and 5′-OH termini suggests that RtcB could have far-reaching practical applications in RNA tagging and cloning.

Materials and Methods.

Expression and purification of Escherichia coli RtcB. The rtcB gene was amplified from E. coli strain MG1655 genomic DNA by PCR using the primers: forward, 5′-TAT GCA TGC ACC ATC ATC ATC ACC ATG GTA ATT ACG AAT TAC TGA CCA C-3′ (SEQ ID NO:1); reverse, 5′-TAT GGA TCC TTA TCC TTT TAC GCA CAC CAC-3′ (SEQ ID NO:2). The PCR product was inserted into the SphI and BamHI sites of a modified pQE-70 vector that encodes Lad for repression of gene expression in the absence of IPTG. After verifying its sequence, the rtcB-containing vector was transformed for expression into Keio strain rtcA, thereby eliminating any contamination from RNA phosphate cyclase (RtcA) during RtcB purification.

Protein was produced by inoculating 0.5 L of Luria-Bertani medium containing ampicillin (100 μg/mL) and growing the culture at 37° C. until OD₆₀₀=0.6. IPTG was then added to 0.5 mM, the temperature was reduced to 32° C., and growth was continued for 2 h. Then, cells were collected by centrifugation. The cell pellet was resuspended in buffer A (50 mM Tris-HCl buffer, pH 7.7, containing 300 mM NaCl, 0.5 mM dithiothreitol (DTT), and 25 mM imidazole), and extracts were prepared by passage through a French pressure cell, followed by centrifugation. The supernatant was loaded onto a column of nickel-nitrilotriacetic acid resin that had been equilibrated with buffer A. The column was washed with 10 column-volumes of buffer A, followed by 10 column-volumes of buffer A containing 40 mM imidazole. The enzyme was eluted with buffer A containing 250 mM imidazole. Purified enzyme was dialyzed against 2 L of 20 mM Tris-HCl buffer, pH 7.4, containing NaCl (200 mM). The concentration of RtcB was determined from the absorbance at 280 nm and a calculated (ExPASy) extinction coefficient of ε₂₈₀=52,370 M⁻¹ cm⁻¹.

Expression and purification of E. coli RtcA. The rtcA gene was amplified from E. coli strain MG1655 genomic DNA by PCR using the primers: forward, 5′-TAT GCA TGC TAA AAA GGA TGA TTG CGC TGG-3′ (SEQ ID NO:3); reverse, 5′-TAT GGA TCC TTC AAT GCT CAC CCG CGT TAC-3′ (SEQ ID NO:4). The PCR product was inserted into the SphI and BamHI sites of a modified pQE-70 vector that encodes LacI for repression of gene expression in the absence of IPTG. After verifying its sequence, the rtcA clone was transformed into E. coli strain BL21 for expression. RtcA was produced, purified, and dialyzed exactly as described for RtcB. The concentration of RtcA was determined from the absorbance at 280 nm and a calculated (ExPASy) extinction coefficient of ε₂₈₀=11,460 M⁻¹ cm⁻¹.

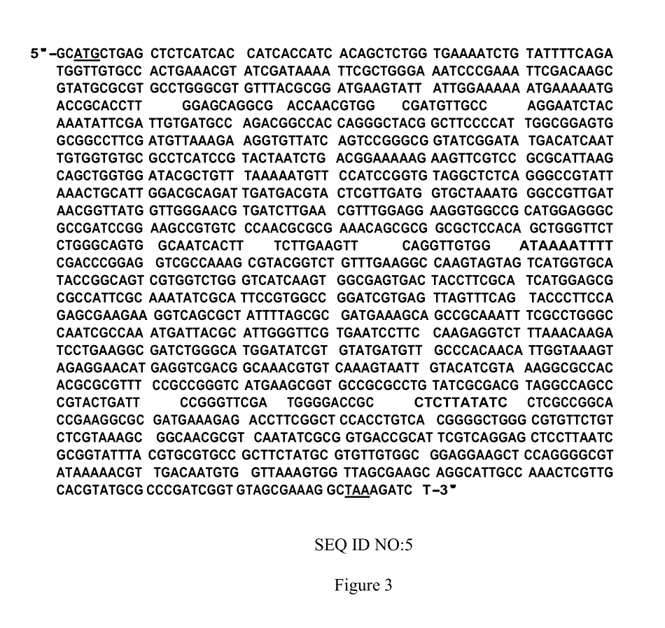

Expression and purification of P. horikoshii RtcB. A gene encoding the 481-residue RtcB enzyme from Pyrococcus horikoshii (SEQ ID NO:10; PDP Accession No. 1UC2) was designed with optimized codons for expression in E. coli (SEQ ID NO:5; see FIG. 3) and synthesized by Integrated DNA Technologies (Coralville, Iowa). The gene also encoded an N-terminal 6× histidine tag and included a 5′ SphI site and a 3′ Bg/II site. P. horikoshii rtcB was cloned into the SphI and Bg/II sites of a modified pQE-70 vector that encodes Lad for repression of gene expression in the absence of IPTG. After verifying its sequence, rtcB-containing vector was transformed into Keio strain rtcA⁻ (FIG. 4), thereby eliminating any contamination from RNA phosphate cyclase (RtcA) during RtcB purification.

P. horikoshii RtcB was produced and purified exactly as described for E. coli RtcB. The stability of the P. horikoshii homologue did, however, require a higher salt concentration and was dialyzed against 20 mM Tris-HCl buffer, pH 7.4, containing NaCl (300 mM). Protein concentrations were determined from absorbance readings at 280 nm using a calculated (ExPASy) extinction coefficient of ε₂₈₀=62,340 M⁻¹ cm¹.

### REFERENCES CITED

1. S. Shuman, C. D. Lima, Curr. Opin. Struct. Biol. 14, 757 (2004).
2. B. Schwer, R. Sawaya, C. K. Ho, S. Shuman, Proc. Natl. Acad. Sci. USA 101, 2788 (2004).
3. J. Popow et al., Science 331, 760 (2011).
4. M. Englert, K. Sheppard, A. Aslanian, J. R. Yates, D. Söll, Proc. Natl. Acad. Sci. USA 108, 1290 (2011).
5. N. Tanaka, S. Shuman, J. Biol. Chem. 286, 7727 (2011).
6. W. Filipowicz, A. J. Shatkin, Cell 32, 547 (1983).
7. K. K. Perkins, H. Furneaux, J. Hurwitz, Proc. Natl. Acad. Sci. USA 82, 684 (1985).
8. J. Abelson, C. R. Trotta, H. Li, J. Biol. Chem. 273, 12685 (1998).
9. R. T. Raines, Chem. Rev. 98, 1045 (1998).
10. S. Xue, K. Calvin, H. Li, Science 312, 906 (2006).
11. K. Calvin, H. Li, Cell. Mol. Life Sci. 65, 1176 (2008).
12. C. M. Cuchillo, M. V. Nogués, R. T. Raines, Biochemistry 50, 7835 (2011).
13. R. Markham, J. D. Smith, Biochem. J. 52, 552 (1952).
14. J. E. Thompson, F. D. Venegas, R. T. Raines, Biochemistry 33, 7408 (1994).
15. W. Filipowicz, M. Konarska, H. J. Gross, A. J. Shatkin, Nucleic Acids Res. 11, 1405 (1983).
16. E. Lund, J. E. Dahlberg, Science 255, 327 (1992).
17. P. Genschik, E. Billy, M. Swianiewicz, W. Filipowicz, EMBO J. 16, 2955 (1997).
18. P. Genschik, K. Drabikowski, W. Filipowicz, J. Biol. Chem. 273, 25516 (1998).
19. O. Vicente, W. Filipowicz, Eur. J. Biochem. 176, 431 (1988).
20. T. Hakoshima, H. Omori, K.-i. Tomita, H. Miki, M. Ikehara, Nucleic Acids Res. 9, 711 (1981).
21. C. Okada, Y. Maegawa, M. Yao, I. Tanaka, Proteins Struct. Funct. Bioinf 63, 1119 (2006).
22. R. Silber, V. G. Malathi, J. Hurwitz, Proc. Natl. Acad. Sci. USA 69, 3009 (1972).
23. C. L. Greer, C. L. Peebles, P. Gegenheimer, J. Abelson, Cell 32, 537 (1983).
24. M. Konarska, W. Filipowicz, H. Domdey, H. J. Gross, Nature 293, 112 (1981).
25. M. Englert, H. Beier, Nucleic Acids Res. 33, 388 (2005).
26. R. M. Izatt, L. D. Hansen, J. H. Rytting, J. J. Christensen, J. Am. Chem. Soc. 87, 2760 (1965).
27. B. Reinhold-Hurek, D. A. Shub, Nature 357, 173 (1992).
28. K. Tomita, T. Ogawa, T. Uozumi, K. Watanabe, H. Masaki, Proc. Natl. Acad. Sci. 97, 8278 (2000).
29. J. Martinez, A. Patkaniowska, H. Urlaub, R. Lührmann, T. Tuschl, Cell 110, 563 (2002).

### Example 2

### Highly Efficient Ligation of RNA Fragments at High Temperature

RtcB from the archaeon, Pyrococcus horikoshii, is highly adept at ligating single-stranded RNA (ssRNA). This enzyme is produced solubly and at high levels in E. coli. The thermostability of this enzyme (P. horikoshii grows optimally at temperatures near 100° C.) allows ligations to be performed optimally at elevated temperatures that denature RNA secondary structure and preclude RNA base pairing.

The ability of P. horikoshii RtcB to ligate ssRNA was demonstrated in ligation reactions that included a 10 nt 5′ RNA fragment and a 10 nt 3′ RNA fragment. The 5′ RNA fragment had a 6-carboxyfluorescein (FAM) label on its 5′ end and was phosphorylated on its 3′ end. The 3′ RNA fragment had hydroxyl groups on each end. The sequence of the 5′ RNA fragment was FAM-5′-AAAUAACAAA-3′-P (SEQ ID NO:6) and the sequence of the 3′ RNA fragment was 5′-AAAUAACAAA-3′ (SEQ ID NO:6). Ligation reactions were performed in 50 μL solutions consisting of 50 mM Tris-HCl buffer, pH 7.4, containing NaCl (500 mM), MnCl₂ (0.5 mM), GTP (100 μM), the two RNA fragments (100 pmol each), RNasin® (20 units), and native P. horikoshii RtcB (4 μM).

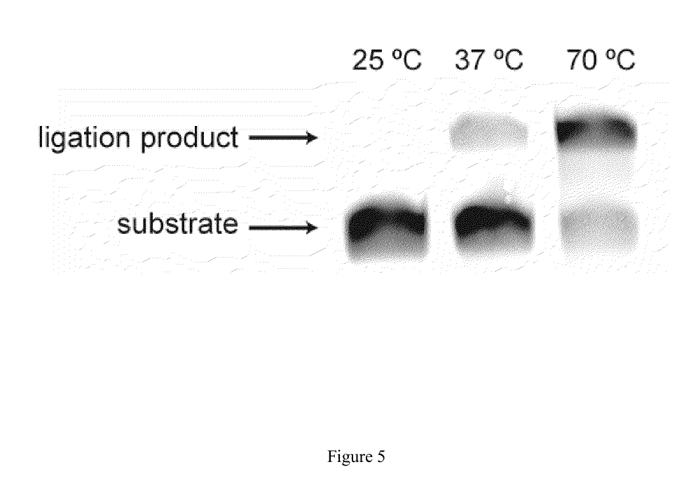

The effect of temperature on the efficiency of the ligation reaction was examined by incubating reaction mixtures at 25, 37, or 70° C. for 2 h, and then adding an equal volume of RNA gel-loading buffer. The reaction mixtures were boiled for 2 min before aliquots (12 μL) were loaded onto an 18% w/v urea-polyacrylamide gel (SequaGel, National Diagnostics, Atlanta, Ga.). Loaded gels were subjected to electrophoresis at 230 V (constant) for 1 h, and then scanned with a Typhoon FLA 9000 imager (GE Healthcare) using the FAM setting. The ligation reaction went to near completion within 2 h at 70° C. (see FIG. 5).

### Example 3

### Ligation of RNA with a 3′-P Terminus to a Library of RNA with 5′-OH Termini

The ability of P. horikoshii RtcB to generate a library of ligated RNA fragments was tested in a ligation reaction that included a 10-nt 5′ RNA fragment (FAM-5′-AAAUAACAAA-3′-P) and a 5-nt (5′-CCCAC-3′), 7-nt (5′-GACAAAU-3′), 10-nt (5′-AAAUAACAAA-3′; SEQ ID NO:6) and 14-nt (5′-UAAAUAACAAAGAU-3′; SEQ ID NO:7) 3′ RNA fragment. Ligation of the 5′ RNA fragment to each of the four 3′ RNA fragments will produce RNA fragments with sizes of 15, 17, 20, and 24 nt.

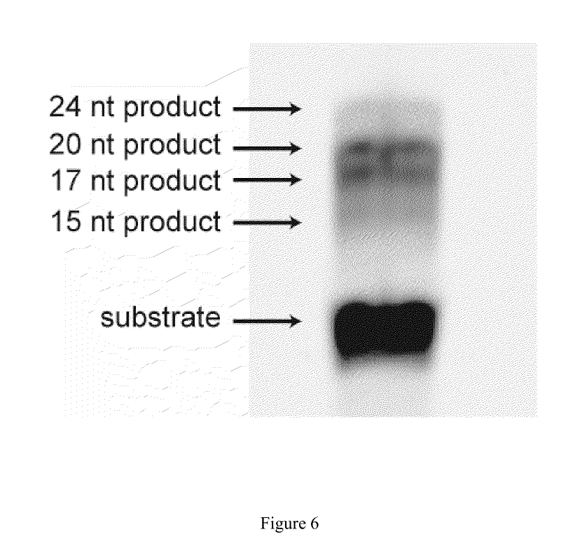

The ligation was performed in a 50 μL solution consisting of 50 mM Tris-HCl buffer, pH 7.4, containing NaCl (500 mM), MnCl₂ (0.5 mM), GTP (100 μM), the five RNA fragments (100 pmol each), RNasin® (20 units), and native P. horikoshii RtcB (4 μM). The reaction mixture was incubated at 70° C. for 2 h, and then an equal volume of RNA gel-loading buffer was added. The reaction mixture was boiled for 2 min before an aliquot (12 μL) was loaded onto an 18% w/v urea-polyacrylamide gel (SequaGel, National Diagnostics, Atlanta, Ga.). Loaded gels were subjected to electrophoresis at 230 V (constant) for 1 h, and then scanned with a Typhoon FLA 9000 imager (GE Healthcare) using the FAM setting. All four possible sizes of RNA ligation products were generated, indicating the capacity of RtcB to create a library of RNA fragments, as shown in FIG. 6.

### Example 4

### Structure-Activity Relationships in RtcB Histidine Guanylation

RtcB catalyzed ligation is presumed to proceed through three nucleotidyl transfer steps. In the first nucleotidyl transfer step, RtcB reacts with GTP to form a covalent RtcB-histidine-GMP intermediate and release PPᵢ; in the second step, the GMP moiety is transferred to the RNA 3′-P; in the third step, the 5′-OH from the opposite RNA strand attacks the activated 3′-P to form a phosphodiester bond and release GMP. Thus, a high-energy phosphoanhydride of GTP is transferred to the nucleic acid 3′-P, activating it for attack by the 5′-OH. In this example we provide insight into the chemical mechanism of an unusual nucleotidyl transfer reaction in this sequence—Mn(II)-dependent histidine guanylylation.

We sought to elucidate the entire pathway of RtcB guanylylation by solving structures of key intermediates. We determined three separate structures of Pyrococcus horikoshii RtcB complexes: (i) a structure with bound Mn(II) represents the intermediate that precedes binding of GTP, (ii) a structure with bound Mn(II) and an unreactive GTP analogue, guanosine 5′-(α-thio)-triphosphate (GTPαS), captures the reaction step immediately preceding formation of the covalent enzyme intermediate, and (iii) a structure of the covalent RtcB-histidine-GMP intermediate depicts the end product of the guanylylation pathway. Our results show that RtcB coordinates a single Mn(II) ion prior to binding GTP and that GTP binds to RtcB in a complex with a second Mn(II) ion. This two-manganese mechanism of RtcB guanylylation is analogous to the two-magnesium mechanism of adenylylation used by canonical ATP-dependent nucleic acid ligases.

Results and Discussion

A Structure with Mn(II) Represents the Intermediate that Precedes GTP Binding. For crystallization of the RtcB/Mn(II) complex, MnCl₂ (1 mM) was added to the concentrated protein solution (200 μM) before crystallization. Crystals of this complex diffracted to a resolution of 2.34 Å, and the apo-RtcB structure (3) was used as a starting model for refinement. The omit density map showed a single Mn(II) ion (Mn1) in a tetrahedral coordination complex with three amino acid residues (Cys98, His234, and His329) and a water molecule.

An earlier crystal structure of RtcB indicated the presence of two manganese ions in the active site, the second manganese ion being coordinated to three amino acid ligands (Asp95, Cys98 and His203) and inorganic sulfate (1). This second site could have been occupied because of the higher Mn(II) ion concentration (3 mM) used in that study. The intracellular concentration of Mn(II) is low (˜0.1 mM) (4), and its inclusion at 1 mM in our study (5× the RtcB concentration) should saturate the relevant coordination sites within the enzyme in the absence of GTP.

A Structure with GTPαS Captures the Step Immediately Preceding RtcB Guanylylation. We reported above that RtcB is unreactive when the GTP analogues guanosine 5′ (α thio)-triphosphate (GTPαS) and guanosine 5′-(α,β-methyleno)-triphosphate (GpCpp) are used as cofactors in ligation reactions, as the bond between the α- and β-phosphoryl groups is recalcitrant to cleavage by RtcB. We chose to pursue structural studies with GTPαS because the modification of a nonbridging oxygen causes minimal perturbation to the phosphoryl groups. To obtain the RtcB/GTPαS/Mn(II) complex, MnCl₂ (2 mM) and GTPαS (1 mM) were added to a concentrated solution of protein (200 μM), and the resulting solution was incubated at 70° C. for 15 min to facilitate any conformational changes in the hyperthermophilic enzyme that are necessary for cofactor binding. Crystals of this complex diffracted to an effective resolution of 2.45 Å (I/σI=2); however, all data to 2.3 Å was used in refinement.

The omit density map of the RtcB/GTPαS/Mn(II) complex indicated the presence of GTPαS and two manganese ions in the RtcB active site. Mn1 remains in tetrahedral coordination geometry with ligands that include the same three amino acid residues as the structure with manganese only; however, the water molecule has been replaced with the nonbridging α-thiophosphate oxygen of GTPαS. A second manganese ion (Mn2) is in tetrahedral coordination geometry with ligands that include a nonbridging oxygen of the γ-phosphoryl group of GTPαS, as well as three amino acid residues (Asp95, Cys98, and His203). The β-phosphoryl group of GTPαS is oriented apically to His404, and its N^{ε} is poised for in-line attack on the α-phosphoryl group (5). Furthermore, H—N^{δ} of His404 forms a hydrogen bond with O^{δ1} of Asp65, which is strictly conserved and appears to orient and activate N^{ε} for attack. A main-chain H—N forms a hydrogen bond with O^{δ2} of Asp65, stabilizing an anti orientation of the carboxylate in the His . . . Asp dyad.

The presence of a cysteine residue bridging two manganese ions in the RtcB active site is sui generis. The Mn1 . . . S and Mn2 . . . S coordination distances in the RtcB/GTPαS/Mn(II) complex are 2.3 Å and 2.4 Å, respectively, and the Mn1 . . . S . . . Mn2 angle is 100°. Thus, the two Mn(II) ions are separated by only 3.6 Å. This distance is similar to the 3.3 Å distance separating the Mn(II) ions of the renowned binuclear manganese cluster in the active site of arginase (6).

RtcB is the only known enzyme catalyzing nucleotidyl transfer that requires a NTP/Mn(II) complex rather than a NTP/Mg(II) complex as a cofactor. The structure of the RtcB/GTPαS/Mn(II) complex provides an explanation for this unusual requirement. First, the ligands of the two bound Mn(II) ions have a tetrahedral geometry, which is disfavored by Mg(II). Second, the side chain of a cysteine residue interacts with both Mn(II) ions, which are more thiophilic than Mg(II) ions. This essential cysteine residue is strictly conserved throughout evolution and likely serves as a gatekeeper that selects for Mn(II) in each metal binding site. Third, Coulombic repulsion deters the close placement of two Mg(II) ions, which have a high charge density. Indeed, the two Mg(II) ions used by T4 RNA ligase are separated by 7.4 Å (2), a distance that is twofold greater than that of the Mn(II) ions in RtcB. More polarizable Mn(II) ions can be accommodated in closer proximity.

An intricate array of hydrogen bonds explains the specificity and high affinity for GTP. The triphosphate moiety forms hydrogen bonds with H—N^{δ} of two asparagine residues. Asn202 has now adopted a different conformation, and its H—N^{δ} forms a hydrogen bond with the β-phosphoryl group. Likewise, H—N^{δ} of Asn330 forms a hydrogen bond with the γ-phosphoryl group. The guanosine nucleoside is bound in an anti conformation with the guanine base stacked on Phe204 and with Tyr451 forming an edge of the guanine-binding pocket. Each carboxylate oxygen of Glu206 forms a hydrogen bond with guanine, one with H—N1 and the other with H—N2; Ser385 also interacts with the H—N2, while H—N^{ε} of Lys480 forms a hydrogen bond with O6. The guanosine ribose 2′- and 3′-oxygens form hydrogen bonds with the main-chain H—N of Ala406 and Gly407, respectively.

The binding of GTPαS elicits significant conformational changes in the RtcB active site. The loop that is displaced by the guanine base has a maximal C^{α} displacement of 2.54 Å at Ser380. In addition, the loop containing Ala406 and Gly407 changes conformation around the ribose 2′-OH and 3′-OH with a maximal C^{α} displacement of 1.37 Å at Ala406.

Structure of the RtcB-Histidine-GMP Covalent Intermediate. To determine the optimal reaction conditions that enable formation of the RtcB-GMP covalent intermediate, ¹⁴C-labeled GTP binding studies were performed (7). The optimal reaction conditions were found to contain purified RtcB (100 μM), GTP (1 mM), and MnCl₂ (2 mM), with incubation at 70° C. for 45 min. Under these conditions, the maximal GMP:RtcB molar ratio was determined to be (0.76±0.02):1. No binding of GTP to RtcB was detected in the absence of Mn(II). Using these reaction conditions, we formed the RtcB-GMP intermediate and removed unbound Mn(II), GTP, and PPᵢ by gel-filtration chromatography. The protein was concentrated to 200 μM, and crystals of this complex diffracted to a resolution of 2.4 Å.

The omit density map indicated the presence of a covalent histidine-GMP and two manganese ions in the RtcB subunit A active site. The GMP density present in subunit B was too weak to model confidently. The guanine and ribose interactions are essentially identical to those in the RtcB/GTPαS/Mn(II) complex. Asn202, however, has shifted to a position near the nascent phosphoramidate bond. The labile histidine-GMP (8) is stabilized by coordination of one nonbridging oxygen of the GMP to Mn1 and the formation of a hydrogen bond of the other nonbridging oxygen with a water molecule. The phosphoimidazolium form of His404 is stabilized by a hydrogen bond from its H—N^{δ} to the carboxylate side chain of Asp65. Mn2 remains bound in tetrahedral coordination geometry; however, the metal contact to the γ-phosphoryl group has been replaced with a water molecule.

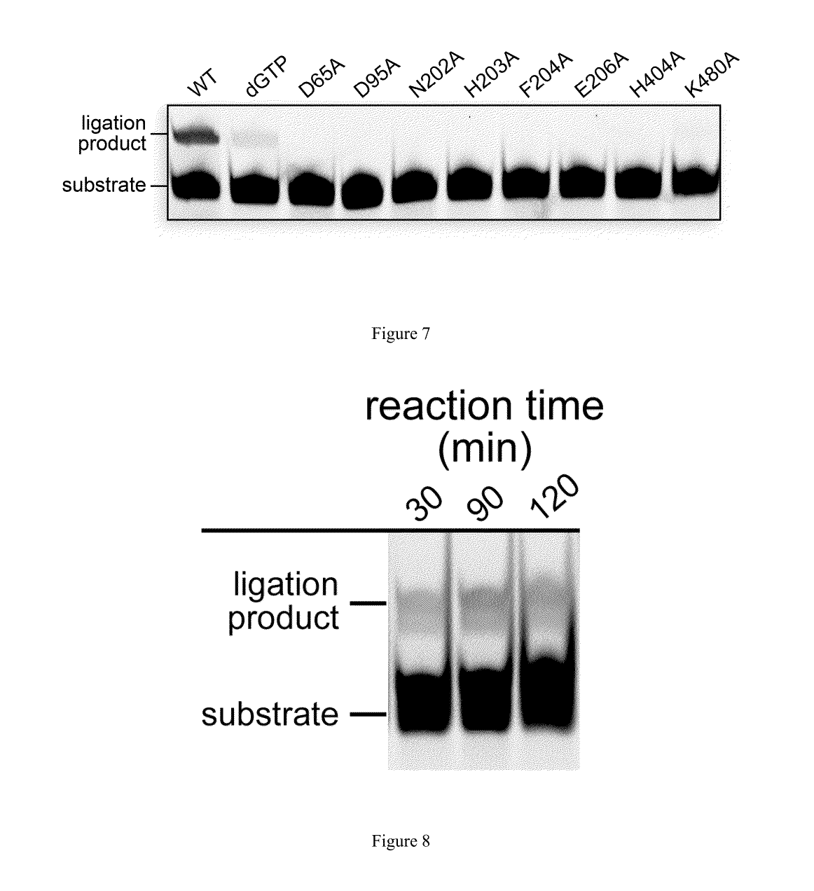

Structure-Guided Mutagenesis of the Guanylate-Binding Pocket. Eight residues of RtcB were found to interact with GMP. Alanine-scanning mutagenesis of these residues revealed their importance for RNA ligation activity (FIG. 7). To assay for RNA ligation, two single-stranded 10-nt RNA fragments were used as substrates in ligation reactions. The 5′ RNA fragment had a 3′-P and a 6-carboxyfluorescein (FAM) label at the 5′ terminus. The 3′ RNA fragment had hydroxyl groups at each terminus. Each of the eight RtcB variants tested were inactive in our ligation assays. The essentiality of these residues is also suggested by their strict evolutionary conservation, with the exception of Phe204, which is substituted with tyrosine is some species. These assays also showed that RtcB can use dGTP as cofactor, although ligation activity is reduced substantially.

RtcB Guanylylation Mechanism. Histidine guanylylation is expected to proceed through an associative mechanism with the accumulation of negative charge on the nonbridging oxygens of the α-phosphoryl group in the pentavalent transition state (5). In the RtcB active site, guanylylation is promoted by neutralization of this negative charge by coordination to Mnl and hydrogen bonds with water molecules. The PPᵢ leaving group of GTP is oriented apically to N^{ε} of His404 by coordination to Mn2. This orientation allows for in-line attack by N^{ε}. The formation of a hydrogen bond between H—N^{δ} of His404 and the side-chain carboxylate of Asp65 orients N^{ε} for attack on the α-phosphorus atom of GTP and stabilizes the phosphoimidazolium group in the ensuing intermediate. Similar hydrogen bonds are common features of other enzymes that are known to proceed through a phosphorylated/nucleotidylated histidine intermediate. In RtcB, the H—N^{δ} proton also serves to make the side chain of His404 into a much better leaving group during the subsequent step in which the GMP moiety is transferred to an RNA 3′-P.

Our structural characterizations show that RtcB and classical ATP-dependent nucleic acid ligases share a similar metal-assisted mechanism for formation of the nucleotidylated enzyme intermediate, despite using a different metal ion. A structure of T4 RNA ligase bound to the ATP analogue adenosine 5′-(α,β-methyleno)-triphosphate (ApCpp) is consistent with a mechanism analogous to the one put forth here for RtcB guanylylation (2). In the T4 RNA ligase structure, a calcium ion is bound in place of one magnesium ion (Mg1) and coordinates to a nonbridging oxygen of the ApCpp Ε-phosphonate and a magnesium ion (Mg2) coordinates to the β-phosphonate group. Mg1 in the T4 ligase structure and Mn1 in the RtcB structure both promote enzyme nucleotidylation by neutralizing the negative charge on the α-phosphoryl group in the pentavalent transition state. The second metal ion observed in both T4 ligase and RtcB enters the active site in a coordination complex with the NTP cofactor and promotes catalysis by orienting the PPᵢ leaving group and neutralizing the charge on the phosphoryl groups. Despite the absence of sequence or structural similarity between RtcB and classical ATP-dependent nucleic acid ligases, Nature has converged on analogous two-metal dependent nucleotidylation mechanisms.

The structures discussed here resolve key issues about RtcB guanylylation. In particular, the structure of the RtcB/GTPαS/Mn(II) complex has revealed the orientation of the bound triphosphate cofactor and the role of Mn2. The consequent molecular description of the two-metal RtcB guanylylation mechanism contrasts with one put forth previously (1), which suggested that GTP enters the RtcB active site without a metal ion complexed to its phosphoryl groups and depicted an incorrect orientation for its β- and γ-phosphoryl groups.

All known nucleotidyl transferases require complexation of a nucleotide triphosphate cofactor to a metal ion. The roles of the metal ion in the NTP/metal complex include orientating the phosphoryl groups, neutralizing their charge, and enhancing their reactivity (9). Accordingly, many enzymes that catalyze the cleavage of a NTP α-β phosphoanhydride bond employ two Mg(II) ions, one that coordinates to the high-affinity site between the β- and γ-phosphoryl groups and a second that coordinates to the α-phosphoryl group. We have revealed how an enzyme can, instead, employ two Mn(II) ions in analogous roles to catalyze nucleotidyl transfer.

Materials and Methods

RtcB Purification. A previously described plasmid expressing P. horikoshii RtcB was used except that the sequence encoding the hexa-histidine tag was removed via mutagenesis (FIG. 3). Native P. horikoshii RtcB was expressed in BL21 cells by growing in Terrific Broth at 37° C. to an OD₆₀₀ of 0.6, inducing with IPTG (0.5 mM) and continuing growth for 3 h. Cells were harvested by centrifugation and resuspended at 8 mL per gram of wet pellet in buffer A (50 mM MES-NaOH, pH 5.6, 45 mM NaCl and 1 mM dithiothreitol). Cells were lysed by passage through a cell disruptor (Constant Systems) at 20,000 psi and the lysate was clarified by centrifugation at 20,000 g for 1 h. Bacterial proteins were precipitated and removed by incubating the lysate at 70° C. for 25 min followed by centrifugation at 20,000 g for 20 min. The clarified lysate was then loaded onto a 5 mL HiTrap HP SP cation-exchange column (GE Lifesciences). The column was washed with 25 mL buffer A, and RtcB was eluted with a NaCl gradient of buffer A (45 mM-1.0 M) over 20 column volumes. Fractions containing RtcB were dialyzed against 4 L of buffer A overnight at 4° C. Dialyzed RtcB was then loaded onto a HiTrap heparin column (GE Lifesciences), and purified RtcB was eluted as described for the cation-exchange chromatography step. Purified RtcB was dialyzed against 4 L of buffer (10 mM HEPES-NaOH, pH 7.5, 200 mM NaCl) overnight at 4° C.

¹⁴C-labeled GTP Binding Assays. Binding assays were performed in 250 μL of 50 mM HEPES buffer, pH 7.5, containing NaCl (200 mM), P. horikoshii RtcB (100 μM), various concentrations of MnCl₂, and [8-¹⁴ C]GTP (Moravek Biochemicals, Brea, Calif.) (7). After incubation, free GTP was removed by applying the reaction to three 5-mL HiTrap desalting columns (GE Lifesciences) connected in series. The desalting columns were equilibrated with elution buffer (50 mM HEPES, pH 7.5, 200 mM NaCl), and protein was eluted in 0.5-mL fractions. Absorbance readings at A₂₆₀ and A₂₈₀ were obtained for each fraction. The protein fractions have high A₂₈₀ readings, whereas the fractions with free GTP have higher A₂₆₀ readings. In fractions containing protein, the RtcB concentrations were calculated from the A₂₈₀ reading using an extinction coefficient of 62,340 M⁻¹ cm⁻¹ (ExPASy). The concentration of [8-¹⁴C]GTP in the protein fractions was determined by liquid scintillation counting. Each 0.5-mL fraction was mixed with 3.5 mL of Ultima Gold MV liquid scintillation cocktail (Perkin Elmer) in a 4-mL vial, and counts were read on a MicroBeta TriLux liquid scintillation counter (Perkin Elmer). The concentration of GTP in each fraction was determined by comparing the counts per minute (cpm) in these samples to the cpm values obtained from standards of known concentration. Optimal formation of the RtcB-GMP complex was found to occur in reaction mixtures that included 1 mM GTP and 2 mM MnCl₂. The optimal incubation conditions were found to be at 70° C. for 45 min. Under these conditions, the GTP:RtcB molar ratio was determined to be (0.76±0.02):1. No binding of GTP to RtcB was detected in the absence of Mn(II).

RtcB Crystallization. RtcB was concentrated to 200 μM (11 mg/mL) by ultrafiltration using a spin concentrator (5,000 MWCO, Amicon) and passed through a 0.2-μm filter. To prepare the RtcB/Mn(II) complex, MnCl₂ (1 mM) was added to the concentrated protein. For preparation of the RtcB/GTPαS/Mn(II) complex, MnCl₂ (2 mM) and a 1:1 mixture of R_{P} and S_{P} diastereomers of GTPαS (1 mM) was added to the concentrated protein, and the resulting solution was incubated at 70° C. for 15 min. For preparation of the RtcB-GMP/Mn(II) complex, the covalent intermediate was formed as described above, and the solution was subjected to gel-filtration chromatography on a Superdex 16/60 column (GE LifeSciences) to remove PPᵢ and excess MnCl₂ and GTP. Each of the protein complexes was flash-frozen in liquid nitrogen. Protein samples were crystallized using the hanging drop vapor diffusion method. Crystals were grown by mixing 1 μL of sample solution with 1 μL of reservoir solution. The RtcB/Mn(II) and RtcB/GTPαS/Mn(II) complexes were crystallized using identical reservoir solutions consisting of Bis-Tris (0.1 M, pH 5.5) and ammonium sulfate (2.1 M), the RtcB-GMP/Mn(II) complex used HEPES-NaOH (0.1 M, pH 7) and ammonium sulfate (2 M). Trays were incubated at 20° C. and crystals appeared within one week. Crystals were harvested and cryoprotected in reservoir solution containing sucrose (20% w/v) and flash-frozen in liquid nitrogen.

Data Collection, Structure Determination and Refinement. X-ray diffraction data were collected at the Life Science Collaborative Access Team at the Advanced Photon Source at Argonne National Laboratory. Datasets were indexed and scaled using HKL2000 (10). The apo-RtcB structure (3) was used as a starting model and the structures were completed using alternating rounds of manual model building using COOT (11) and refinement with phenix.refine (12). Structure quality was assessed by MolProbity (13) and figures were generated using PyMOL (14). The GMP in the RtcB-GMP structure was fitted into the difference density and refined using phosphoramidate bond distance and angle values derived from the small-molecule X-ray crystal structure of 1-carboxymethyl-2-imino-3-phosphonoimidazolidine (15). Omit maps were calculated using Phenix.

RNA Ligation Assay. Ligation assays with single-stranded RNA as the substrate used two 10-nt oligonucleotides that were synthesized by Integrated DNA Technologies (15). The 5′ RNA fragment had a 6-carboxyfluorescein (FAM) label on its 5′ end and was phosphorylated on its 3′ end. The 3′ RNA fragment had hydroxyl groups on each end. The sequence of the 5′ fragment was FAM-5′-AAAUAACAAA-3′-P (SEQ ID NO:6) and the sequence of the 3′ RNA fragment was 5′-AAAUAACAAA-3′ (SEQ ID NO:6). Ligation reactions were performed in 10-μL solutions consisting of 50 mM Bis-Tris buffer, pH 7.0, containing NaCl (300 mM), MgCl₂ (0.25 mM), GTP (100 μM), each RNA fragment (1 μM), and RtcB (10 μM). Reaction mixtures were incubated at 70° C. for 1 h prior to the addition of water (40 μL) and RNA-loading buffer (50 μL). Reaction mixtures were subjected to electrophoresis and visualized as previously described in the previous examples.

### REFERENCES CITED

1. Englert M, et al. (2012) Structural and mechanistic insights into guanylylation of RNA-splicing ligase RtcB joining RNA between 3′-terminal phosphate and 5′-OH. Proc Natl Acad Sci USA 109:15235-15240.
2. El Omani, et al. (2006) Molecular architecture and ligand recognition determinants for T4 RNA ligase. J Biol Chem 281:1573-1579.
3. Okada C, Maegawa Y, Yao M, Tanaka I (2006) Crystal structure of an RtcB homolog protein (PH1602-extein protein) from Pyrococcus horikoshii reveals a novel fold. Proteins 63:1119-1122.
4. Finney L A, O'Halloran T V (2003) Transition metal speciation in the cell: Insights from the chemistry of metal ion receptors. Science 300:931-936.
5. Lassila J K, Zalatan J G, Herschlag D (2011) Biological phosphoryl-transfer reactions: Understanding mechanism and catalysis. Annu Rev Biochem 80:669-702.
6. Kanyo Z F, Scolnick C R, Ash D E, Christianson D W (1996) Structure of a unique binuclear manganese cluster in arginase. Nature 383:554-557.
7. Morar M, Anand R, Hoskins A A, Stubbe J, Ealick S E (2006) Complexed structures of formylglycinamide ribonucleotide amidotransferase from Thermotoga maritime describe a novel ATP binding protein superfamily. Biochemistry 45:14880-14895.
8. Hultquist D E (1968) The preparation and characterization of phosphorylated derivatives of histidine. Biochim Biophys Acta 153:329-340.
9. Cleland W W, Hengge A C (2006) Enzymatic mechanisms of phosphate and sulfate transfer. Chem Rev 106, 3252-3278.
10. Otwinowski Z, Minor W (1997) Processing of X-ray diffraction data collected in oscillation mode. Methods Enzymol 276:307-326.
11. Emsley P, Cowtan K (2004) Coot: Model-building tools for molecular graphics. Acta Crystallogr D Biol Crystallogr 60:2126-2132.
12. Adams P D, et al. (2010) PHENIX: A comprehensive Python-based system for macromolecular structure solution. Acta Crystallogr D Biol Crystallogr 66:213-221.
13. Davis I W, et al. (2007) MolProbity: All-atom contacts and structure validation for proteins and nucleic acids. Nucleic Acids Res 35:W375-383.
14. Delano W L (2002) The PyMOL Molecular Graphics System (DeLano Scientific, San Carlos, Calif.).
15. Phillips G N Jr, Thomas J W Jr, Annesley T M, Quiocho F A (1979) Stereospecificity of creatine kinase. Crystal structure of 1-carboxymethyl-2-imino-3-phosphonoimidazolidine. J Am Chem Soc 101:7120-7121.

### Example 5

### Generation of DNA/RNA Hybrid Molecules with RtcB

We tested the ability of RtcB to ligate DNA to RNA. Ligation reaction mixtures included a 10-nt 5′ DNA/RNA hybrid fragment and a 10 nt 3′ RNA fragment. The 5′ nucleic acid fragment contained 9 DNA bases and 1 RNA nucleotide as the terminal residue on the 3′ end. This 5′ nucleic acid fragment also had a 6-carboxyfluorescein (FAM) label on its 5′ end and was phosphorylated on its 3′ end. The 3′ RNA fragment had hydroxyl groups on each end. The sequence of the 5′ fragment was FAM-5′-d(AAAUAACAA)A-3′-P (SEQ ID NO:8), and the sequence of the 3′ RNA fragment was 5′-AAAUAACAAA-3′ (SEQ ID NO:6). Ligation reactions were performed in 50 μL solutions consisting of 50 mM Tris-HCl buffer, pH 7.4, containing NaCl (300 mM), MnCl₂ (0.25 mM), GTP (100 μM), 100 pmoles of each RNA fragment, and native P. horikoshii RtcB (5 μM). The results of this ligation reaction are depicted in FIG. 8. RtcB was able to generate a DNA/RNA hybrid fragment. It is also anticipated that RtcB can use a DNA substrate as the 3′ nucleic acid fragment.

### Example 6

### Archease Activates RtcB and Expands its NTP Cofactor Specificity

A. Production and Purification of Pyrococcus horikoshii Archease.

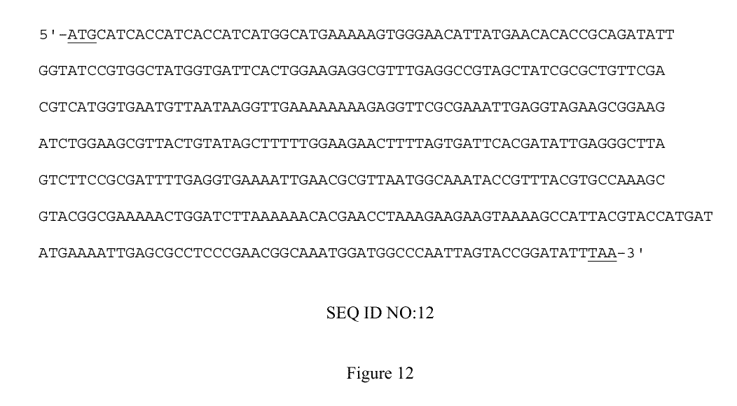

The gene encoding Archease is localized adjacent to genes encoding proteins that are involved in DNA or RNA processing, suggesting that Archease functions as a modulator or chaperone of these proteins. We synthesized a gene encoding Pyrococcus horikoshii Archease (SEQ ID NO:9; UniProt Accession No. O59205) using codons optimized for expression in Escherichia coli (SEQ ID NO:12, see FIG. 12). This gene also encoded an N-terminal hexahistidine tag. The gene was cloned between the SphI and BamHI restriction enzyme sites of the vector pQE-70-lacI.

The resulting plasmid was transformed into the BL21 strain of E. coli, and the Archease protein was produced by growing cells in Terrific Broth to an OD₆₀₀ of 0.6, inducing with 0.5 mM IPTG, and continuing growth for 3 hours at 37° C. Cells were harvested by centrifugation and resuspended in 8 mL per gram of wet pellet in buffer A (50 mM Tris-HCl, pH 7.7, 300 mM NaCl, 25 mM imidazole, and 1 mM dithiothreitol). Cells were lysed by passage through a cell disruptor (Constant Systems) at 20,000 psi, and the lysate was clarified by centrifugation at 20,000 g for 1 h. Bacterial proteins were precipitated and removed by incubating the lysate at 70° C. for 25 min followed by centrifugation at 20,000 g for 20 min. The clarified lysate was loaded onto a 5-mL HiTrap nickel-nitrilotriacetic acid column (GE Lifesciences). The column was washed with 50 mL of buffer A, and then 50 mL of buffer A containing 50 mM imidazole. Archease was eluted with buffer A containing 250 mM imidazole. Purified Archease was dialyzed against 2 L of buffer (25 mM Tris-HCl, pH 7.5, 100 mM NaCl, and 10% glycerol) overnight at 4° C. and protein was flash-frozen in liquid nitrogen.

B. Archease Enhances the Catalytic Activity of RtcB.

The ability of Archease to activate P. horikoshii RtcB was tested in reactions using ssRNA substrates. Ligation reactions included a 10-nt 5′ RNA fragment and a 10-nt 3′ RNA fragment. The 5′ RNA fragment had a 6-carboxyfluorescein (FAM) label on its 5′ end and was phosphorylated on its 3′ end. The 3′ RNA fragment had hydroxyl groups on each end. The sequence of the 5′ fragment was FAM-5′-AAAUAACAAA-3′-P (SEQ ID NO:6) and the sequence of the 3′ RNA fragment was 5′-AAAUAACAAA-3′ (SEQ ID NO:6). Ligation reactions were performed in 50-μL solutions consisting of 50 mM Tris-HCl buffer, pH 7.4, containing NaCl (300 mM), MnCl₂ (0.25 mM), GTP (100 μM), RNA fragments (100 pmol of each), and P. horikoshii RtcB (5 μM).

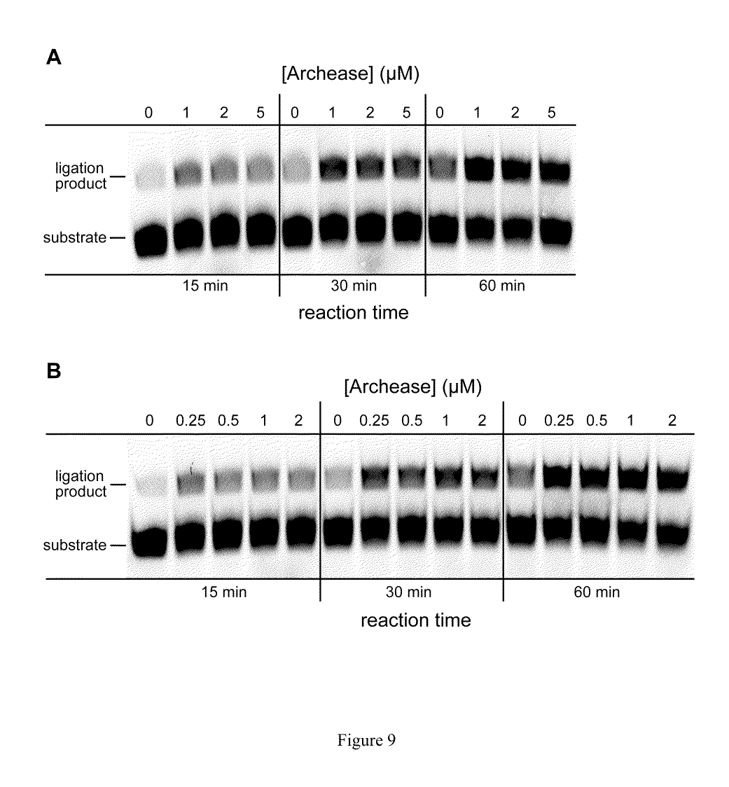

Various concentrations of Archease were added to these ligation reactions to test its effect. As depicted in FIG. 9, the addition of Archease at concentrations that are lower than that of RtcB or RNA enhanced catalysis of RNA ligation. These results indicate that Archease significantly enhances the catalytic activity of RtcB.

C. Archease Enables RtcB to use dGTP Efficiently as a Cofactor.

RtcB alone can use dGTP as a cofactor, but at a rate that is much lower than that with GTP. We hypothesized that if Archease is activating RtcB by facilitating the loading of GTP, then Archease would also enable activation of RtcB when dGTP is used in ligation reactions. Furthermore, when the 2′-OH at the ligation junction that is vicinal to the 3′-P is replaced with a 2′-F, the rate of the ligation reaction is reduced substantially. We hypothesized that if Archease activates RtcB by increasing the rate of the ligation step, then it would increase the ligation rate of substrate RNA having a 2′-F.

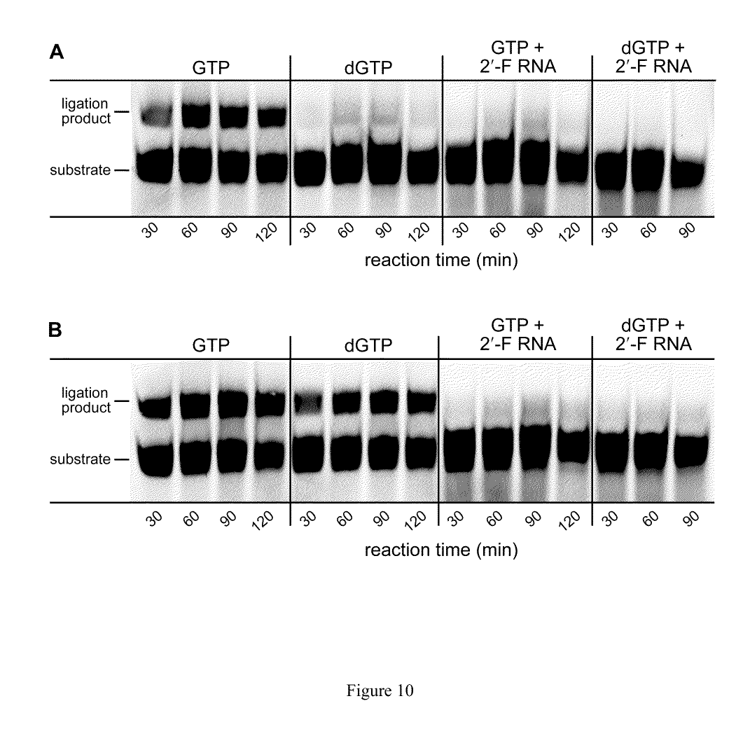

Ligation reaction mixtures were set up as described above, and each was divided into two tubes. No Archease was added to one set of reaction mixtures (FIG. 10A), and Archease was added to a concentration of 0.25 μM to the other set (FIG. 10B). As depicted in FIG. 10, Archease selectively activated reactions in which either GTP or dGTP were used as cofactors. Archease had no effect on ligation reactions that used 2′-F RNA. These results indicate that Archease enables RtcB to use dGTP efficiently as a cofactor.

D. Archease Expands the NTP Cofactor Specificity of RtcB to Include ATP

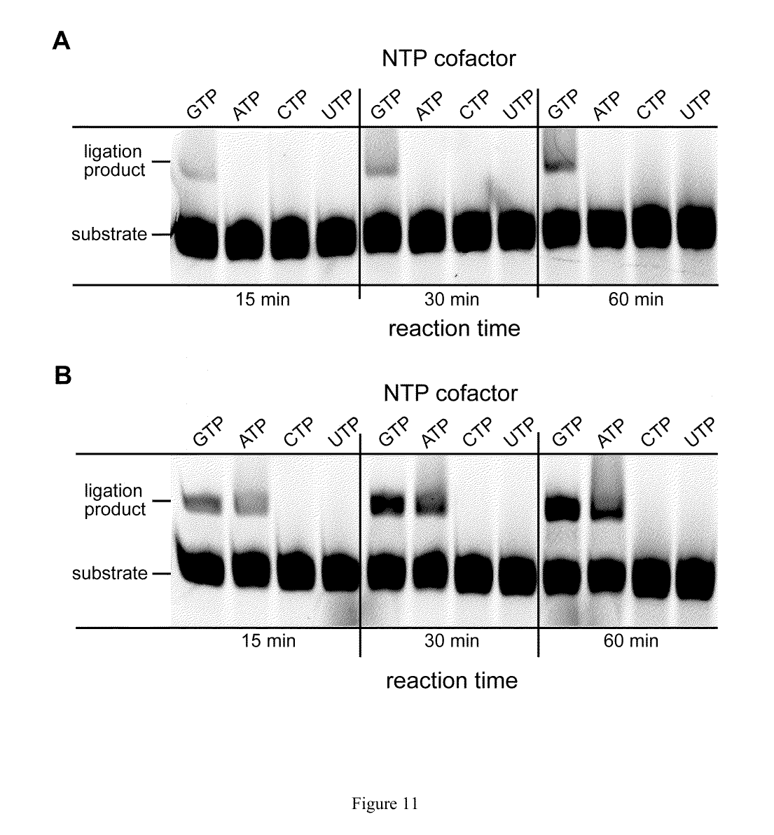

We tested the ability of Archease to enable RtcB to use cofactors other than GTP or dGTP. Ligation reaction mixtures with RtcB were set up as described above, and each was divided into two tubes. No Archease was added to one set of reaction mixtures (FIG. 11A), and Archease was added to a concentration of 0.25 μM to the other set (FIG. 11B). The various NTPs were included at a concentration of 100 μM. As depicted in FIG. 11, Archease enables RtcB to use ATP but not CTP or UTP as a cofactor in ligation reactions.

Those skilled in the art will recognize, or be able to ascertain using no more than routine experimentation, numerous equivalents to the specific materials and methods described herein. Such equivalents are considered to be within the scope of this invention and encompassed by the following claims.

We claim:

1. A method for covalently joining a first nucleic acid molecule and a second nucleic acid molecule, wherein the first nucleic acid molecule comprises a 3′-phosphate terminus wherein the nucleotide at the 3′-phosphate terminus is an RNA nucleotide, and wherein the second nucleic acid molecule comprises a 5′-hydroxyl terminus, the method comprising the step of:
contacting the 3′-phosphate terminus of the first nucleic acid and the 5′-hydroxyl terminus of the second nucleic acid molecule with a 2′,3′-cyclic phosphate RNA ligase (RtcB) in the presence of manganese (II) ion (Mn²⁺) and a purine triphosphate, whereby the 3′-phosphate terminus of the first nucleic acid molecule and the 5′-hydroxyl terminus of the second nucleic acid molecule are covalently joined.

2. The method of claim 1, wherein one or more of the RtcB, the first nucleic acid molecule, the second nucleic acid molecule, and the purine triphosphate are isolated.

3. The method of claim 1, wherein the method is performed in vitro.

4. The method of claim 1, wherein the purine triphosphate is guanosine-5′-triphosphate (GTP) or deoxyguanosine-5′-triphosphate (dGTP).

5. The method of claim 1, wherein the method is performed in the presence of an Archease.

6. The method of claim 5, wherein the purine triphosphate is adenosine-5′-triphosphate (ATP).

7. The method of claim 1, wherein the method is performed in the absence of RNA phosphate cyclase (RtcA).

8. The method of claim 1, wherein the first nucleic acid molecule is an RNA molecule or a DNA molecule having at least one RNA nucleotide at the 3′-phosphate terminus, and wherein the second nucleic acid molecule is an RNA molecule or a DNA molecule.

9. The method of claim 1, wherein the first and second nucleic acid molecules are the same molecule, and wherein a circular nucleic acid molecule is formed.

10. The method of claim 1, wherein the second nucleic acid molecule comprises a detection moiety.

11. The method of claim 1, wherein the method is performed at a temperature above 50° C.

12. A method for making a library of nucleic acid fragments, comprising contacting a first nucleic acid molecule comprising a 3′-phosphate terminus wherein the nucleotide at the 3′-phosphate terminus is an RNA nucleotide and a plurality of second nucleic acid molecules having a 5′-hydroxyl terminus with a 2′,3′-cyclic phosphate RNA ligase (RtcB), manganese (II) ion (Mn²⁺), and a purine triphosphate, whereby a library of nucleic acid fragments is made.

13. The method of claim 12, wherein the purine triphosphate is guanosine-5′-triphosphate (GTP), deoxyguanosine-5′-triphosphate (dGTP), or adenosine-5′-triphosphate (ATP), with the proviso that if the purine triphosphate is ATP, the method is performed in the presence of an Archease.

14. A kit for covalently joining a first nucleic acid molecule comprising a 3′-phosphate terminus, wherein the nucleotide at the 3′-phosphate terminus is an RNA nucleotide, and a second nucleic acid molecule having a 5′-hydroxyl terminus, the kit comprising:
(a) an isolated 2′,3′-cyclic phosphate RNA ligase (RtcB); and
(b) either:
(i) an isolated guanosine-5′-triphosphate (GTP) or deoxyguanosine-5′-triphosphate (dGTP); or
(ii) an isolated Archease.

15. The kit of claim 14, wherein the kit comprises an isolated Archease, and wherein the kit further comprises an isolated purine triphosphate.

16. The kit of claim 15, wherein the purine triphosphate is GTP, dGTP, or adenosine-5′-triphosphate (ATP).

17. The kit of claim 14, wherein the kit further comprises a composition comprising manganese (II) ion.

18. The kit of claim 14, further comprising a nucleic acid molecule having a 5′-hydroxyl terminus.

19. The kit of claim 18, wherein the nucleic acid molecule having a 5′-hydroxyl terminus comprises a detection moiety.

20. The kit of claim 19, wherein the nucleic acid molecule having a 5′-hydroxyl terminus is an RNA molecule or a DNA molecule.
